(19) United States
(12) Patent Application Publication
(10) Pub. No.: US 2012/0016152 A1
(43) Pub. Date: Jan. 19, 2012

(54) Process for the preparation of 4`-haloalkylbiphenyl-2-carboxylic acids

(75) Inventors: Michael Kreis, Leverkusen (DE); Alain Cotte, Leverkusen (DE); Antje Steinhaus, Koln (DE); Lukas Gooßen, Kaiserslautern (DE); Thomas Knauber, Kaiserslautern (DE)

(73) Assignee: SALTIGO GMBH, Langenfeld (DE)

(21) Appl. No.: 13/049,038

(22) Filed: Mar. 16, 2011

(30) Foreign Application Priority Data
Mar. 20, 2010 (DE) 102010012133.9

(51) Int. Cl. C07C 69/78; C07C 63/331

(52) U.S. Cl. 560/102; 562/492

(57) ABSTRACT

The invention relates to a process for the preparation of optionally substituted 4′-haloalkyl-biphenyl-2-carboxylic acid esters and to a process for the preparation of corresponding optionally substituted 4′-haloalkylbiphenyl-2-carboxylic acids.

The invention relates to a process for the preparation of optionally substituted 4′-haloalkyl-biphenyl-2-carboxylic acid esters and to a process for the preparation of corresponding optionally substituted 4′-haloalkylbiphenyl-2-carboxylic acids.

4′-Haloalkylbiphenyl-2-carboxylic acids are important intermediates for the manufacture of drugs. In particular, 4′-trifluoromethylbiphenyl-2-carboxylic acid itself is known as a pharmaceutical active ingredient under the name Xenalipin. Xenalipin lowers the cholesterol content and the fraction of triglycerides in blood plasma (Arteriosclerosis, 1987, 64, 27-35). 4′-Tritluoromethylbiphenyl-2-carboxylic acid is therefore a drug for preventing cardiac and circulatory disorders. 4′-haloalkylbiphenyl-2-carboxylic acids can be prepared in a manner known per se by saponifying 4′-haloalkylbiphenyl-2-carboxylic acid esters.

JP 2004-067595 A discloses a process for the preparation of a 4′-haloalkylbiphenyl-2-carboxylic acid ester, in particular of isopropyl 4′-trifluoromethylbiphenyl-2-carboxylate, by nickel-catalysed coupling of isopropyl 2-chlorobenzoate and 4-chlorobenzotrifluoride. This process has the disadvantage that stoichiometric amounts of nickel have to be used and only small yields are achieved.

Processes for the preparation of 4′-haloalkylbiphenyl-2-carboxylic acids, in particular of 4′-trifluoromethylbiphenyl-2-carboxylic acid, by means of Suzuki-Miyaura coupling of alkylboronic acid with the corresponding aryl iodides, aryl bromides or aryl chlorides are known from DE 19963563 A, Tetrahedron Letters, 47, 2006, 4225-4229 and Synthesis, 8, 2002, 1043-1046. However, this approach has serious disadvantages, especially for industrial applications. The boronic acids are prepared in complex syntheses from highly reactive, air- and moisture-labile organometallic compounds (e.g. Grignard reagents or organolithiums) and trialkyl borates, where many functional groups are not tolerated. These include carboxylic acids and derivatives thereof. The process can therefore not be used economically in industrial processes and, moreover, is unacceptable in terms of safety.

Further processes for the preparation of 4″-haloalkylbiphenyl-2-carboxylic acids by catalytic cross-couplings of aryl halides with diboron compounds are known from CN 1944386 and EP 1122234 A. Disadvantages of these processes are that starting materials which are unacceptable in terms of safety are used, the processes involve at least four process steps and only low yields are achieved.

EP 1223158 describes a process for the preparation of 4′-haloalkylbiphenyl-2-carboxylic acids, in particular of 4′-trifluoromethylbiphenyl-2-carboxylic acid, starting from 2-cyano-4-methylbiphenyl via chlorination, fluorination and subsequent saponification. Disadvantages of this process are that the starting materials have to be prepared in a complex manner and that the process proceeds inefficiently in industrial operations.

A four-stage process for the preparation of 4′-trifluoromethylbiphenyl-2-carboxylic acid via a Negishi coupling starting from benzoic acid is known from Journal of Labelled Compounds and Radiopharmaceuticals 31, 1992, 1011-1017 and from Journal Organic Preparations and Procedures International 27, 1995, 367-372. On account of the highly reactive organolithium compound used, the process is unacceptable in terms of safety and necessitates the use of 2-phenyl-4,4-dimethyl-2-oxazoline as protected and expensive benzoic acid equivalent, meaning that, moreover, the process cannot be used economically in industrial operations.

It is known from U.S. Pat. No. 4,578,522 B that 4′-trifluoromethylbiphenyl-2-carboxylic acid can be prepared starting from 2-(2-methoxyphenyl)-4,4-dimethyl-2-oxazoline and 4-bromo-benzotrifluoride in a Wurtz-Grignard reaction with subsequent saponification. A disadvantage of this process is that only low yields are achieved.

It is common to all of the processes that they are either unsuitable for reasons of safety and cost, or the target molecule can only by synthesized via synthesis involving many stages and only low yields are achieved.

There therefore continues to be a need for a process for the preparation of optionally substituted 4′-haloalkylbiphenyl-2-carboxylic acids with which the disadvantages of the prior art can be overcome and with which optionally substituted 4′-haloalkyl-2-biphenylcarboxylic acids can be prepared in good yields and efficiently in industrial operations.

Surprisingly, a process has been found in which firstly the 4′-haloalkylbiphenyl-2-carboxylic acid esters can be prepared in good yields and high purities starting from optionally substituted phthalic anhydride by metal-catalysed decarboxylating cross-coupling. The 4′-haloalkylbiphenyl-2-carboxylic acid esters can then be converted to the 4′-haloalkylbiphenyl-2-carboxylic acids by saponification.

The invention therefore relates to a process for the preparation of the compounds of the formula (I)

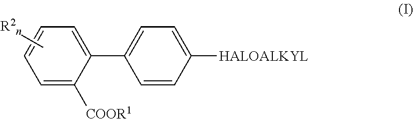

where R¹ is C₁-C₆-alkyl, C₆-C₂₄-aryl or C₇-C₁₅-arylalkyl, and R² may be independent of the others or identical and is C₁-C₆-alkyl, C₂-C₆-alkenyl, C₃-C₁₆-heteroaryl, C₁-C₆-alkoxy, C₁-C₆-aryloxy, C₁-C₆-acyl, C₇-C₁₅-arylalkyl, C₁-C₈-mono- and dialkylamino, nitro, cyano, C₁-C₆-alkylthio, C₆-C₂₄-aryl, a 3- to 7-membered saturated or partially unsaturated heterocycle or hydrogen and when R² is a C₃-heteroaryl, the C₃-heteroaryl contains three carbon atoms and at least two nitrogen atoms or one nitrogen atom and one oxygen atom or one nitrogen atom and one sulphur atom, and n=1, 2, 3 or 4, and HALOALKYL is a straight-chain, cyclic or branched C₁-C₆-alkyl radical which is mono-, poly- or completely halogenated, i.e. substituted by F, Cl, Br and/or I,
in which compounds of the formula (II)

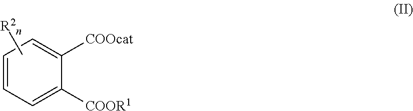

in which R¹, R² and n have the aforementioned meaning and cat is an inorganic or organic singly charged cation,
in the presence of at least one copper source,
in the presence of at least one cyclic, chelating amine,
in the presence of at least one phosphine ligand and
in the presence of at least one palladium source
are reacted with compounds of the formula (n)

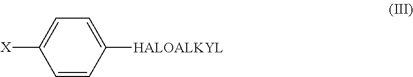

in which HALOALKYL has the aforementioned meaning and X is halogen=Cl, Br, I, pseudohalogen, arylsulphonate or alkylsulphonate, to give compounds of the formula (I).

Preferably, R¹ is methyl, ethyl, s-propyl, n-propyl, n-, s-, tert-butyl, neopentyl, cyclohexyl, benzyl or phenyl. Very particularly preferably, R¹ is isopropyl or s-propyl.

Preferably, R² is C₁-C₆-alkyl, C₂-C₆-alkenyl, C₃-C₁₆-heteroaryl, C₁-C₆-acyl, C₆-C₂₄-aryl and/or hydrogen. Particularly preferably, R² is methyl, ethyl, s-, n-propyl, n-, s-, tert-butyl and/or hydrogen. Very particularly preferably, R² is hydrogen.

n is preferably 1 or 2, very particularly preferably n=1. If n=1, R² is preferably C₁-C₆-alkyl, C₂-C₆-alkenyl, C₃-C₁₆-heteroaryl, C₆-C₂₄-aryl, C₁-C₆-acyl and/or hydrogen. Very particularly preferably, R² is then a C₁-C₆-alkyl, C₂-C₆-alkenyl and/or hydrogen.

If n=2, 3 or 4, R² may be identical or different and is then preferably C₁-C₆-alkyl, C₂-C₆-alkenyl, C₃-C₁₆-heteroaryl, C₆-C₂₄-aryl, C₁-C₆-acyl, and/or hydrogen. Very particularly preferably, R² is then a C₁-C₆-alkyl, C₂-C₆-alkenyl and/or hydrogen.

HALOALKYL is a C₁-C₆-haloalkyl. HALOALKYL is preferably dichloromethyl, difluoromethyl, chlorodifluoromethyl, fluoromethyl, 2,2,2-trifluoroethyl, difluoroethyl, fluoroethyl, fluoropropyl, tetrafluoroethyl, heptafluoropropyl or a C₁-C₆-perhaloalkyl, which is a C₁-C₆-alkyl radical completely substituted by halogen atoms. By way of example and preferably, C₁-C₆-perhaloalkyl is trifluoromethyl, trichloromethyl, tribromomethyl, pentafluoroethyl, heptafluoropropyl, cyclo-nonafluoropentyl, cyclononachlorocyclopentyl, heptafluoroisopropyl and nonafluorobutyl. Preferably, HALOALKYL is a C₁-C₆-perfluoroalkyl. Preferably, C₁-C₆-perfluoroalkyl is difluoromethyl, trifluoromethyl, pentafluoroethyl, heptafluoroisopropyl and nonafluorobutyl. Particularly preferably, HALOALKYL and/or C₁-C₆-perhaloalkyl is trifluoromethyl, pentafluoroethyl, trichloromethyl, tribromomethyl or pentachloroethyl. Very particularly preferably, HALOALKYL is trifluoromethyl, pentafluoroethyl or heptafluoroisopropyl.

Cat is preferably an alkali metal cation or an organic cation from the series ammonium, pyridinium or phosphonium. Particularly preferably, cat is a potassium cation.

The copper sources used are inorganic or organic copper(I) or copper(II) compounds or elemental copper. The copper(I) compounds or copper(II) compounds used may be copper(I) halides, such as e.g. copper(I) bromide or copper(I) chloride or copper(II) halides, such as e.g. copper(II) bromide or copper(II) chloride or copper(I) pseudohalides, such as e.g. copper(I) thiocyanate, copper(I) isothiocyanate, copper(I) isocyanate or copper(I) cyanate or copper(II) pseudohalides, such as e.g. copper(II) thiocyanate, copper(II) isothiocyanate or copper(II) cyanate or copper(I) cyanide or copper(II) cyanide or organic copper(I) complexes or organic copper(II) complexes, such as e.g. dichloro(1,10-phenanthroline)copper(II), dibromo(1,10-phenanthroline)copper(II) or copper(II) diamine complexes, such as e.g. di-μ-hydroxybis(N,N,N′,N′-tetramethylethylenediamine)copper(II) chloride or copper(I) oxide or copper(II) oxide or mixtures of these compounds. As copper sources, preference is given to using copper(I) halides, copper(II) halides or copper(II) oxide. Very particularly preferably, the copper source used is copper(I) chloride or copper(II) oxide.

The palladium sources used are inorganic or organic Pd(I) or Pd(II) compounds or elemental Pd. Particular preference is given to using palladium(II) compounds and even more preferably palladium(II) sulphate, palladium(II) halides, such as palladium(II) bromide or palladium(II) chloride, palladium(II) pseudohalides, such as palladium(II) thiocyanate, palladium(II) isothiocyanate, palladium(H) cyanate or palladium(II) isocyanate, or palladium(II) cyanide, or organic palladium(II) compounds, such as palladium(II) carboxylate, such as palladium(II) acetate, palladium(II) formate, palladium(II) propylate or palladium(II) butylate or palladium(II) acetylacetonate, or palladium(II) complexes such as dichloro(N,N,N′,N′-tetramethyl-ethylenediamine)palladium(II), dichloro(1,10-phenanthroline)palladium(II), bis(triphenyl-phosphine)palladium(II), bis(benzonitrile)palladium(II), 1,4-bis(diphenylphosphino)butanepalladium(II) chloride, (2,2′-bipyridine)dichloropalladium(II), (1,3-bis(diphenylphosphino)propane)palladium(I) chloride, or mixtures of these compounds. Very particular preference is given to using palladium(II) acetylacetonate as palladium source.

Chelating, cyclic amines which can be used are, for example and preferably, substituted, cyclic aromatic diamines, such as e.g. phenanthroline, bipyridine, terpyridine and substituted derivatives of these compounds. Particularly preferably, phenanthroline, bipyridine, terpyridine and substituted derivatives of these compounds are used as cyclic, chelating amines. Very particular preference is given to using 1,10-phenanthroline as cyclic, chelating amine.

The phosphine ligands used are compounds of the formula (IV),

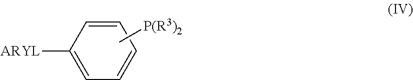

where ARYL is a C₆-C₂₄-aryl unsubstituted or substituted one or more times, independently of one another, by C₁-C₆-alkyl, C₁-C₆-alkoxy, sulphonates or C₁-C₈-mono- or dialkylamino, and R³ is a C₁-C₆-alkyl radical or a C₆-C₂₄-aryl unsubstituted or substituted one or more times.

Preferably, ARYL is a phenyl radical which may optionally be polysubstituted, independently of one another, by C₁-C₆-alkyl, C₁-C₆-alkoxy, sulphonates or C₁-C₈-mono- or dialkylamino. Very particularly preferably, ARYL is a phenyl radical which may be substituted one or more times by methoxy, tert-butyl, sulphonates or dimethylamino.

R³ is preferably a methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl, n-hexyl, benzyl, o-, p-, m-tolyl, o-, p-, m-hydroxytolyl or o-, p-, m-methoxybenzyl. Particularly preferably, R³ is tert-butyl or cyclohexyl.

Very particularly preferably, the phosphine ligands are 2-(di-tert-butylphosphino)biphenyl, dicyclohexylphosphino-2-biphenyl, 2-dicyclohexylphosphino-2′-(N,N-dimethylamino)biphenyl 2-dicyclohexylphosphino-2′,6′-dimethoxybiphenyl, 2-dicyclohexylphosphino-2′,4′,6′-triisopropylbiphenyl, dicyclohexylphosphino-2′-methylbiphenyl 2-di-tert-butylphosphino-2′,4′,6′-triisopropyl-biphenyl, 2-di-tert-butylphosphino-3,4,5,6-tetramethyl-2′,4′,6′-triisopropyl-1,1′-biphenyl, 2-di-tert-butylphosphino-2′-methylbiphenyl, 2-di-tert-butylphosphino-2′-(N,N-dimethylamino)biphenyl, 2-dicyclohexylphosphino-2′-(N,N-dimethylamino)biphenyl, 2-(dicyclohexylphosphino)biphenyl, 2-(di-tert-butylphosphino)biphenyl, sodium 2-dicyclohexylphosphino-2′,6′-dimethoxybiphenyl-3′-sulphonate, 2-diphenylphosphino-2′-(N,N-dimethylamino)biphenyl or 2-dicyclohexylphosphino-2′,6′-diisopropoxybiphenyl.

X is preferably Cl, Br, I, F, N₂⁺, triflate, tosylate, nonaflate or mesylate. Particularly preferably, X is Cl, triflate, tosylate, nonaflate or mesylate. Very particularly preferably, X is Cl.

The use of a copper(II) oxide with a 1,10-phenanthroline in combination with a palladium(II) acetylacetonate and a phosphine ligand has proven to be particularly preferred. In particular, it is further preferred that the phosphine ligand used is a sterically exacting electron-rich phosphine ligand, such as e.g. 2-dicyclohexylphosphinobiphenyl.

The scope of the invention encompasses all of the radical definitions, parameters and explanations above and below, specified generally or in preferred ranges, among one another, i.e. also between the respective ranges and preferred ranges in any desired combination.

Alkyl or alkenyl or alkoxy is in each case independently a straight-chain, cyclic or branched alkyl or alkenyl or alkoxy radical. The same applies to the nonaromatic moiety of an arylalkyl radical.

C₁-C₆-alkyl is, for example, methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Preferably, C₁-C₆-alkyl is methyl, ethyl, n-propyl, isopropyl, n-butyl, sec-butyl, tert-butyl, n-pentyl, 1-methylbutyl, 2-methylbutyl, 3-methylbutyl, neopentyl, 1-ethylpropyl, cyclohexyl, cyclopentyl and n-hexyl.

By way of example and preferably, C₂-C₆-alkenyl is vinyl, allyl, isopropenyl and n-but-2-en-1-yl. C₁-C₆-Alkoxy is, for example, methoxy, ethoxy, n-propoxy, isopropoxy, n-butoxy, sec-butoxy and tert-butoxy, n-pentoxy, 1-methylbutoxy, 2-methylbutoxy, 3-methylbutoxy, neopentoxy, 1-ethylpropoxy, cyclohexoxy, cyclopentoxy and n-hexoxy.

C₁-C₆-Alkylthio is a straight-chain, cyclic or branched alkylthio radical having 1 to 6 carbon atoms. Preference is given to a straight-chain or branched alkylthio radical having 1 to 4, particularly preferably having 1 to 3, carbon atoms. By way of example and preferably, mention may be made of: methylthio, ethylthio, n-propylthio, isopropylthio, tert-butylthio, n-pentylthio and n-hexylthio.

Within the context of the invention, C₆-C₂₄-aryl is a mono-, bi- or tricyclic carbocyclic aromatic radical having preferably 6 to 24 aromatic carbon atoms. Furthermore, the carbocyclic aromatic radicals can be substituted with up to five identical or different substituents per cycle, selected from the group C₁-C₆-alkyl, C₇-C₁₅-arylalkyl, C₆-C₂₄-aryl, C₁-C₈-mono- and dialkylamino, nitro, cyano or 3- to 7-membered saturated or partially unsaturated heterocycle. By way of example and preferably, aryl is biphenyl, phenyl, o-, m-, p-tolyl, naphthyl, phenanthrenyl, anthracenyl, acetnaphthylene and fluorenyl.

C₇-C₁₅-Arylalkyl is in each case, independently of the others, a straight-chain, cyclic or branched alkyl radical according to the above definition which may be substituted once, several times or completely by aryl radicals according to the above definition. For example and preferably, C₇-C₁₅-arylalkyl is benzyl.

Within the context of the invention, arylsulphonates are a C₆-C₂₄-aryl radical or a C₇-C₁₅-arylalkyl radical according to the aforementioned definition which is substituted by one or more sulphonic acid groups and is linked via an oxygen atom of the sulphonic acid group. By way of example and preferably, arylsulphonates are tosylate or benzylate.

Within the context of the invention, alkylsulphonates are a C₁-C₆-alkyl radical according to the aforementioned definition which is substituted by one or more sulphonic acid groups and is linked via an oxygen atom of the sulphonic acid group. By way of example and preferably, alkylsulphonates are mesylate, triflate or nonaflate.

Within the context of the invention, 3- to 7-membered saturated or partially unsaturated heterocycle is a heterocycle with up to 3 identical or different heteroatoms from the series S, N and/or O, which is linked via a ring carbon atom or a ring nitrogen atom and which can contain one or two double bonds. Preference is given to a 5- to 7-membered saturated heterocycle with up to 2 identical or different heteroatoms from the series S, N and/or O. By way of example, mention may be made of: tetrahydrofur-2-yl, tetrahydrofur-3-yl, pyrrolidin-1-yl, pyrrolidin-2-yl, pyrrolidin-3-yl, pyrrolin-1-yl, piperidin-1-yl, piperidin-4-yl, 1,2-dihydropyridin-1-yl, 1,4-dihydropyridin-1-yl, piperazin-1-yl, morpholin-4-yl, thiomorpholin-4-yl, azepin-1-yl, 1,4-diazepin-1-yl. Preference is given to piperidinyl, piperazinyl, morpholinyl and pyrrolidinyl.

Within the context of the invention, C₃-C₁₆-heteroaryl is an aromatic heterocycle having up to 3 identical or different heteroatoms from the series S, N and/or O, which is linked via a ring carbon atom of the heteroaromatic, optionally also via a ring nitrogen atom of the heteroaromatic and which has between 3 and 16 carbon atoms (C₃-C₁₆-heteroaryl), preferably 3 to 7 (C₃-C₇) carbon atoms and particularly preferably 4 to 5 (C₄-C₅) carbon atoms (C₄-C₅-heteroaryl). C₃-C₁₆-heteroaryl, C₃-C₇-heteroaryl and C₃-C₅-heteroaryl always have at least enough heteroatoms for the heteroaromatic to be aromatic. A C₃—N-heteroaryl thus has three carbon atoms and at least two nitrogen atoms. C₃-C₁₆-heteroaryl can be further substituted by radicals selected from the group C₁-C₆-alkoxy, C₇-C₁₅-arylalkyl, C₁-C₈-mono- and dialkylamino, nitro, cyano, C₁-C₆-alkylthio, C₆-C₂₄-aryl or 3- to 7-membered saturated or partially unsaturated heterocycle. By way of example and preferably, the following may be mentioned as C₃-C₁₆-heteroaryl: pyridyl, pyrimidyl, pyridazinyl, pyrazinyl, thienyl, furyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl, benzofuranyl or dibenzofuranyl.

Within the context of the invention, C₁-C₈-mono- or dialkylamino is an amino group with one or two identical or different, cyclic, straight-chain or branched alkyl substituents, which preferably in each case have 1 to 8 carbon atoms.

By way of example and preferably, C₁-C₈-monoalkylamino is methylamino, ethylamino, n-propylamino, isopropylamino, t-butylamino, n-pentylamino and n-hexylamino.

By way of example and preferably, C₁-C₈-dialkylamino is N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Pseudohalogen refers, within the context of the invention to substituents which, in terms of their chemical properties, are very similar to the halogens. These are e.g. sulphonates and halosulphonates, such as e.g. tosylate, triflate and nonafluorobutylsulphonate, but also thiocyanate, cyanate, isothiocyanate, isocyanate and azide. Preferably, pseudohalogens are thiocyanate, cyanate, isothiocyanate, isocyanate and azide.

The process according to the invention for the preparation of the compounds of the formula (I) can be carried out in the presence of a solvent or without dilution. Preferably, the process is carried out without dilution. For example and preferably, the starting materials used may serve as solvents, or linear, cyclic and branched hydrocarbons, such as, for example, hexanes, heptanes and octanes, aromatic hydrocarbons, such as, for example, benzene, toluene, xylenes, ethylbenzene, mesitylene, ethers, such as, for example, 1,4-dioxane, tetrahydrofuran, methyltetrahydrofuran, dibutyl ether, methyl t-butyl ether, diisopropyl ether, diethylene glycol dimethyl ether, esters, such as, for example, ethyl acetate, butyl acetate, amides such as, for example, dimethylformamide, diethylformamide, N-methylpyrrolidone, dimethylacetamide or dimethyl sulphoxide or sulpholane or nitriles such as, for example, acetonitrile, isobutyronitrile, propionitrile or propylene carbonate or chlorinated aliphatic and aromatic hydrocarbons or mixtures of these solvents. Particular preference is given to using dimethylformamide, diethylformamide, N-methylpyrrolidone, mesitylene, dimethylacetamide, dimethyl sulphoxide, sulpholane, acetonitrile and propylene carbonate or mixtures of these solvents. Very particular preference is given to using N-methylpyrrolidone, mesitylene or mixtures of these two solvents.

The process according to the invention for the preparation of the compounds of the formula (I) is carried out for example at temperatures between 100° C. and 300° C., preferably at temperatures between 140° C. and 250° C., particularly preferably at temperatures between 160° and 210° C.

The process according to the invention is generally carried out at atmospheric pressure. In general, the process can be carried out at any desired pressure.

According to the process of the invention, the copper and palladium sources and the cyclic, chelating amines are used, independently of one another, in amounts of from 0.001 mol % to 100 mol %, based on the compounds of the formula (III), preference being given to using amounts of from 0.001 mol % to 10 mol % and particular preference being given to using amounts of from 0.01 mol % to 6 mol %, based on the compounds of the formula (III).

The copper sources and the palladium sources are moreover used preferably in a quantitative ratio of from 10 (copper source):1 (palladium source) up to 1:2, particularly preferably in a quantitative ratio of from 10:1 to 1:1.

In the process according to the invention for the preparation of the compounds of the formula (I), the quantitative ratios of the compounds of the formula (II) and of the compounds of the formula (III) are between 4:1 and 1:10, preferably between 2:1 and 1:5 and particularly preferably the quantitative ratios of the compounds of the formula (II) and of the compounds of the formula (III) are between 2:1 and 1:2.

The phosphine ligands and/or the cyclic, chelating amines can likewise be added in the form of complex compounds of the copper and/or palladium sources. In this case, an additional addition of phosphine ligands and/or cyclic, chelating amines is not necessarily required. This type of reaction procedure is likewise encompassed by the process according to the invention.

The process according to the invention for the preparation of the compounds of the formula (I) from the compounds of the formula (II) is carried out in an essentially water-free manner. Essentially water-free means that the water content, based on the amount of the reaction mixture used, is preferably between 0.0001% by weight and 1.0% by weight.

As a rule, the process according to the invention for the preparation of the compounds of the formula (I) is carried out such that firstly the copper source, the cyclic, chelating amine, the palladium source, the phosphine ligand and the compounds of the formula (II) are introduced as initial charge and then the solvent is added. Then, the reaction mixture is generally rendered inert. Then, e.g. the compounds of the formula (III) are added and the reaction mixture is heated. The end of the reaction can be determined using analytical methods known to the person skilled in the art, such as e.g. chromatographically. The products are likewise worked-up by customary methods known to the person skilled in the art, such as e.g. by means of extraction with organic solvents and/or distillation. The addition of the starting materials explained above can likewise take place in a different order.

Preferably, the process according to the invention for the preparation of the compounds of the formula (I) is carried out such that first the copper source, the cyclic, chelating amine, the palladium source and the compounds of the formula (II) are introduced as initial charge and then the solvent or the solvent mixture is added. Preferably, the reaction mixture is then rendered inert, e.g. by flushing with nitrogen. The compounds of the formula (III) are then added and the reaction mixture is heated. The end of the reaction is determined by means of gas chromatography. The products are likewise worked-up by customary methods known to the person skilled in the art, such as e.g. by means of extraction with organic solvents and/or distillation.

Also encompassed by the invention is a process for the preparation of the compounds of the formula (II),

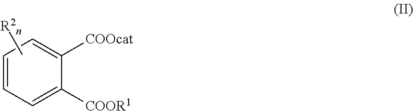

where R¹ is C₁-C₆-alkyl, C₆-C₂₄-aryl or C₇-C₁₅-arylalkyl and
R² may be independent of the others or identical and is C₁-C₆-alkyl, C₂-C₆-alkenyl, C₃-C₁₆-heteroaryl, C₁-C₆-alkoxy, C₁-C₆-aryloxy, C₁-C₆-acyl, C₇-C₁₅-arylalkyl, C₁-C₈-mono- and dialkylamino, nitro, cyano, C₁-C₆-alkylthio, C₆-C₂₄-aryl, 3- to 7-membered saturated or partially unsaturated heterocycle or hydrogen, and when R² is a C₃-heteroaryl, the C₃-heteroaryl contains three carbon atoms and at least two nitrogen atoms or one nitrogen atom and one oxygen atom or one nitrogen atom and one sulphur atom and n=1, 2, 3 or 4 and cat is an inorganic or organic singly charged cation, where compounds of the formula (V)

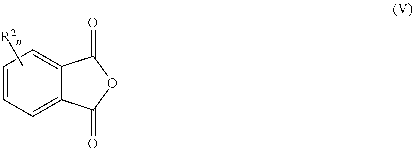

in which R² and n have the aforementioned meaning,
are reacted in a step a) with a compound of the formula (VI)

R¹(OH)ₘ (VI)

where R¹ has the aforementioned meaning and m may be 1, 2, 3 or 4,
to give compounds of the formula (VII)

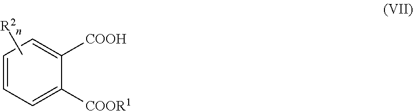

and in a step b) the compounds of the formula (VII) are reacted with at least one base, consisting of one or more singly charged, inorganic or organic cations and corresponding anions, to give compounds of the formula (II).

Bases within the context of the process according to the invention for the preparation of the compounds of the formula (II) from the compounds of the formula (V) are preferably alkali metal hydroxides or alkylates, such as e.g. sodium methylate, potassium tert-butylate, sodium hydroxide, potassium hydroxide or organic bases, such as e.g. pyridine, ammonium compounds, such as e.g. ammonium hydroxide or phosphonium compounds or mixtures of these bases. Particularly preferably, potassium hydroxide is used as base. Within the context of the process according to the invention for the preparation of the compounds of the formula (II) from the compounds of the formula (V), cat is the cation corresponding to these bases.

Preferably, m=1 or 2, particularly preferably m=1.

Compounds of the formula (VI) for the purposes of the invention are mono-, di- or trihydric, aliphatic or aromatic alcohols. As compounds of the formula (VI), preference is given to using aliphatic, cyclic or noncyclic branched or unbranched alcohols. As compounds of the formula (VI) particular preference is given to using benzene, phenol, methanol, ethanol, n- or isopropanol, n-, s-, i,- or tert-butanol, neopentanol or cyclohexanol, very particular preference being given to using iso- or s-propanol as compounds of the formula (VI).

The process according to the invention for the preparation of the compounds of the formula (II) can be carried out in the presence of an organic solvent. Preferably, the process is carried out without dilution. Particularly preferably, the compounds of the formula (VI) are used in excess such that the compounds of the formula (VI) can be used as solvents and as starting materials. If the process is carried out in the presence of an organic solvent, any organic inert solvent is suitable for mixing with the compounds of the formula (VI). Suitable organic, inert solvents in the process according to the invention are in particular polar, organic solvents, such as e.g. ketones, nitriles or sulphones.

Step a) of the process according to the invention for the preparation of the compounds of the formula (II) takes place, for example and preferably, at temperatures at which the compounds of the formula (VI) boil. Step a) of the process according to the invention can be carried out at various pressures. Preferably, step a) of the process according to the invention for the preparation of the compounds of the formula (II) is carried out at standard pressure.

The compounds of the formula (VI) used in step a) of the process according to the invention for the preparation of the compounds of the formula (II) are used, for example and preferably, in molar excess relative to the compounds of the formula (V). Preferably, the compounds of the formula (V) and the compounds of the formula (VI) are used in a quantitative ratio greater than 1:5, particularly preferably in a quantitative ratio greater than 1:10.

The bases used in step b) of the process according to the invention for the preparation of the compounds of the formula (II) and the compounds of the formula (VII) are used, for example, in a quantitative ratio of from 0.1 to 10, preferably from 0.1 to 5, and particularly preferably in a quantitative ratio of from 0.8 to 1.2.

Step b) of the process according to the invention for the preparation of the compounds of the formula (II) can be carried out, for example, at room temperature under standard pressure.

Step a) of the process according to the invention for the preparation of the compounds of the formula (II) is preferably carried out such that the compounds of the formula (V) are mixed with the compounds of the formula (VI) and then heated. The end of the reaction can be determined by gas chromatography. The compounds of the formula (VII) are then isolated and further reacted to give the compounds of the formula (II). This takes place by dissolving compounds of the formula (VII) in a solvent and adding the bases to them, preferably in metered form. Other orders of addition are also possible in principle.

In one particular embodiment of the invention, the preparation according to the invention of the compounds of the formula (II) takes place in a one-pot process, in particular without further purification of the compounds of the formula (VII). This means that step b) is carried out in step a) and consequently step b) can be dispensed with.

In a further particularly preferred embodiment, the preparation of the compounds of the formula (I) takes place in a one-pot process, in particular without further purification of the compounds of the formula (II). In this embodiment, the compounds of the formula (V) are generally firstly introduced as initial charge. The compounds of the formula (VI) can then be added. The end of the reaction can be determined e.g. by means of gas chromatography. Then, for example the base is added. After the end of the reaction, e.g. the resulting water is separated off, for example by distillation. Then, for example, the solvent, the copper source, the cyclic, chelating amine, the phosphine ligand, the palladium source and then the compounds of the formula (III) can be added. The system can then for example be rendered inert, e.g. by flushing the reaction mixture with nitrogen or argon. It could then for example be heated. The end of the reaction can be determined e.g. by means of gas chromatography. Work-up takes place by customary methods known to the person skilled in the art, e.g. by means of extraction or distillation.

Preferably, the compounds of the formula (V) are initially introduced and the compounds of the formula (VI) added. The end of the reaction is determined by analytical ways known to the person skilled in the art. The base is then added and the resulting water is removed, preferably by distillation. The solvent, the compounds of the formula (III), the copper source, the cyclic, chelating amine, the phosphine ligand and the palladium source are then added, rendered inert and then heated. The end of the reaction is determined by gas chromatography.

If the process according to the invention for the preparation of the compounds of the formula (II) is carried out in a one-pot process, the quantitative ratios of the compounds of the formula (V) and of the bases are for example between 0.1 and 10, preferably between 0.1 and 5 and particularly preferably between 0.8 and 1.2.

The compounds of the formula (I) are 4′-haloalkylbiphenyl-2-carboxylic acid esters. From the compounds of the formula (I), it is possible to prepare, by saponification with an acid or a base, the compounds of the formula (VIII)

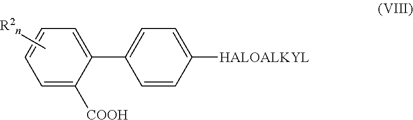

in which R², n and HALOALKYL have the meanings given for the compounds of the formula (I).

A process for the preparation of the compounds of the formula (VIII) from the compounds of the formula (I) is therefore also encompassed by the invention. The compounds of the formula (VIII) can likewise be present in the form of their salts and/or are prepared in the form of these salts from the compounds of the formula (I). Acids or bases which can be used for the preparation of the compounds of the formula (V111) from the compounds of the formula (I) are sulphuric acid, nitric acid, hydrohalic acids or alkali metal hydroxides.

The preparation of the compounds of the formula (ya) from the compounds of the formula (I) can be carried out at temperatures between 20° C. and 200° C., preferably between 30° C. and 100° C.

Solvents which can be used for the preparation of the compounds of the formula (VIED from the compounds of the formula (I) are all water-miscible organic inert solvents, such as e.g. inert amides, such as, for example and preferably, dimethylformamide, diethylformamide, N-methylpyrrolidone or dimethylacetamide.

In general, the preparation of the compounds of the formula (VII) from the compounds of the formula (I) can be carried out according to customary conditions, known to the person skilled in the art, for the saponification of a carboxylic acid ester.

The feed materials and starting materials used in the processes according to the invention can either be prepared by processes known to the person skilled in the art or are commercially available.

With the help of the process according to the invention it is possible to prepare the compounds of the formula (I) in high yields and economically in industrial operations. The by-products that are formed are of little risk from an ecological point of view. Moreover, the process according to the invention has advantages from safety-related aspects. The process for the preparation of the compounds of the formula (I) can be carried particularly efficiently in a one-pot process, without further purification of the compounds of the formula (II) or of the compounds of the formula (VII), with compounds of the formula (V) as starting materials. The compounds of the formula (I) can be readily converted by saponification to the compounds of the formula (VIII), which can thus be prepared as required.

The compounds of the formula (VIII) and thus also the compounds of the formula (I) are important intermediates for pharmaceutical active ingredients which are suitable e.g. for treating cardiovascular disorders. Some of the compounds of the formula (VIII), in particular 4′-trifluoromethylbiphenyl-2-carboxylic acid, are suitable as drugs.

The examples below serve to illustrate the invention and should not be interpreted as a limitation.

### EXAMPLES

### 1. Preparation of Monoisopropyl Phthalate

Phthalic anhydride (14.8 g, 100 mmol) was suspended in ⁱPrOH (isopropanol) (50 ml) in a 100 ml round-bottomed flask with reflux condenser and heated at boiling for 6 h. After cooling the clear solution to room temperature, it was concentrated by evaporation and the remaining colourless oily residue was dried in vacuo, during which it slowly crystallized out and the product (20.8 g, 99%) was obtained as a colourless solid. ¹H-NMR (600 MHz, CDCl₃) D=12.46 (s, 1H), 7.85 (d, ³J=7.6 Hz, 1H), 7.64 (d, ³J=7.6 Hz, 1H), 7.54 (t, ³J=7.6 Hz, 1H), 7.49 (t, ³J=7.6 Hz, 1H), 5.24 (h, ³J=6.2 Hz, 1H), 1.32 (d, ³J=6.7 Hz, 6H) ppm. ¹³C-NMR (151 MHz, CDCl₃)=172.6, 167.5, 133.7, 132.0, 130.4, 129.6, 129.5, 128.5, 69.5, 21.3 ppm.

### 2. Preparation of Potassium Monoisopropylphthalate

Monoisopropyl phthalate (10.4 g, 50.0 mmol) was dissolved in 2-propanol (20 ml) in a 250 ml round-bottomed flask. A solution of potassium hydroxide (3.30 g, 50.0 mmol) in 2-propanol was added dropwise at room temperature over the course of 30 min. When the addition was complete, the mixture was stirred for a further half an hour, then the solvent was removed and the remaining colourless solid was taken up in methyl tert-butyl ether (100 ml). The suspension was stirred for 15 min, during which a voluminous colourless solid precipitated out, which was filtered off, washed with methyl tert-butyl ether (3×50 ml) and dried in vacuo. The salt (9.82 g, 80%) was obtained as colourless solid. ¹H-NMR (400 Mz, D₂O) δ=7.64 (d, ³J=7.5 Hz, 1H), 7.42-7.52 (m, 2H), 7.36 (t, ³J=7.5 Hz, 1H), 5.09 (h, ³J=6.2 Hz, 1H), 1.29 (d, ³J=6.2 Hz, 6H) ppm. ¹³C NMR (101 MHz, D₂O)=176.6, 169.9, 141.0, 132.3, 129.2, 128.8, 128.8, 127.4, 70.8, 21.3 ppm.

### 3. Preparation of Potassium Mononeopentylphthalate

Neopentyl alcohol (25.0 g, 284 mmol) was weighed into a 250 ml round-bottomed flask and heated to 70° C., during which the alcohol melted. Potassium tert-butylate (5.61 g, 50.0 mmol) was added and the mixture was stirred until a clear, colourless solution was obtained. Phthalic anhydride (7.41 g, 50.0 mmol) was added, stirring was carried out for 10 min and the colourless difficult-to-stir reaction mass was cooled to room temperature, suspended in ethanol and washed with ethanol, filtered and dried in vacuo. The product (13.1 g, 95%) was obtained as a colourless solid. ¹H-NMR (400 MHz, MeOH-D4) δ=7.84-7.86 (m, 1H), 7.59-7.62 (m, 2H), 7.47-7.52 (m, 1H), 4.08 (s, 2H), 1.13 (s, 9H) ppm. ¹³C NMR (101 MHz, MeOH-D4) δ=176.8, 170.0, 143.5, 132.8, 130.5, 130.0, 129.1, 128.7, 76.0, 32.3, 27.2 ppm.

### 4. Preparation of Potassium Monobenzylphthalate

At 70° C., potassium tert-butylate (11.2 g, 100 mmol) was dissolved in benzyl alcohol (50.0 g, 462 mmol) in a 500 ml round-bottomed flask. Powdered phthalic anhydride (14.8 g, 100 mmol) was added to the clear colourless solution and stirred for 1 h at 70° C. The solvent was removed and the remaining colourless, waxy residue was washed several times with ethanol and dried in vacuo. The product (28.9 g, 98%) was obtained as a colourless solid. ¹H-NMR (400 MHz, DMSO-D6) δ=7.72 (d, ³J=7.5 Hz, 1H), 7.44 (d, ³J=7.2 Hz, 2H) 7.27-7.39 (m, 6H), 5.21 (s, 2H) ppm. ¹³C NMR (101 MHz, DMSO-D6) δ=170.2, 169.0, 141.1, 136.5, 133.0, 129.2, 128.7, 128.3, 127.9, 127.7, 127.5, 126.2, 66.0 ppm.

### 5. Preparation of Isopropyl 4′-trifluoromethylbiphenyl-2-carboxylate

Copper(I) chloride (5.94 mg, 0.06 mmol), 1,10-phenanthroline (10.8 mg, 0.06 mmol), Pd(acac)₂ (palladium(II) acetylacetonate) (1.83 mg, 0.06 mmol), 2-(di-tert-butylphosphino)biphenyl (John-Phos) (5.37 mg, 0.018 mmol) and potassium monoisopropylphthalate (493 g, 2.00 mmol) were weighed into a 20 ml headspace vial, dried for 30 min in vacuo and back-filled with nitrogen, and a freshly degassed solution of mesitylene and N-methylpyrrolidone (4.0 ml, 3:1) and 4-chlorobenzotrifluoride (361 g, 2.00 mmol, 2677 μl) was added. The orange reaction mixture was stirred at 170° C. for 16 h. After cooling to room temperature, the red-brown reaction solution was filtered and washed with ethyl acetate and then with aqueous HCl, extracted again with ethyl acetate and then dried. The remaining brown oil was purified by means of flash chromatography (silica gel, n-hexane/ethyl acetate, 9:1) and the product (409 mg, 66%) was obtained as a colourless oil. ¹H-NMR (600 MHz, CDCl₃) δ=7.92 (d, ³J=7.9 Hz, 1H), 7.66 (d, ³J=8.2 Hz, 2 H), 7.52 (t, ³J=7.4 Hz, 1H), 7.40-7.46 (m, 3H), 7.31 (d, ³J=7.7 Hz, 1H), 5.01 (h, ³J=6.2 Hz, 1H), 1.04 (d, ³J=6.2 Hz, 6H) ppm. ¹³C-NMR (151 MHz, CDCl₃) δ=167.4, 145.4, 140.9, 131.3, 131.1, 130.3, 129.9, 129.3, 129.1, 128.8, 127.7, 125.1, 124.7 (q), 123.3, 68.5, 21.1 ppm.

### 6. Preparation of Isopropyl 4′-trifluoromethylbiphenyl-2-Carboxylate in a One-Pot Process

Phthalic anhydride (35.4 g; 0.24 mol) was suspended in 120 ml of isopropanol in a 0.5 l sulphonation beaker with precision-ground glass stirrer, combination condenser (can be used as high-efficiency condenser and as distillation bridge as desired) and dropping funnel and then stirred for 6 h at 60° C. The resulting solution was cooled to room temperature and a solution of KOH in isopropanol (15.8 g in 120 ml) was added and stirred for a further hour. N-methylpyrrolidone was then added, and isopropanol and the water which was formed were removed by distillation. The resulting solution was admixed with 270 ml of mesitylene, 28.8 g of 4-chlorobenzotrifluoride, 0.8 g of CuCl, 1.6 g 1,10-phenanthroline, 0.8 g palladium acetate and 2.2 g of dicyclohexylphosphine-2-biphenyl (dicyclohexyl-Johnphos). The reaction mixture was evacuated and then aerated with nitrogen and heated to 170° C. The reaction solution was then stirred at 170° C. for 24 h, cooled and filtered over activated carbon. Then, firstly mesitylene was distilled off and the residue was taken up in isopropyl acetate. The organic phase was admixed with 10% strength hydrochloric acid, extracted and the phase was separated. The solvents were firstly removed from the organic phase by distillation under reduced pressure, and then the product was also distilled. This gave 34.4 g (0.11 mol, 70%) of isopropyl 4′-trifluoromethylbiphenyl-2-carboxylate as a colourless oil. ¹H-NMR (400 MHz, CDCl₃) δ=7.92 (d, 3J=7.9 Hz, 1H), 7.66 (d, 3J=8.2 Hz, 2H), 7.52 (t, 3J=7.4 Hz, 1H), 7.40-7.46 (m, 3H), 7.31 (d, 3J=7.7 Hz, 1H), 5.01 (h, 3J=6.2 Hz, 1H), 1.04 (d, 3J=6.2 Hz, 6H) ppm.

### 7. Preparation of Isopropyl 4′-trifluoromethylbiphenyl-2-carboxylate

Potassium monoisopropylphthalate (26.6 g, 108 mmol), copper(II) oxide (358 mg, 4.50 mmol), 1,10-phenanthroline (811 mg, 4.50 mmol), Pd(acac)₂ (137 mg, 0.45 mmol) and cyclohexyl-John-Phos (315 mg, 0.90 mmol) were weighed into an oven-dried 250 ml three-neck flask with reflux condenser and dried for 1 h in vacuo. The apparatus was back-filled with nitrogen, and a solution, degassed with argon, of dry mesitylene/NMP (180 ml, 3:1) and 4-chlorobenzotrifluoride (16.6 g, 92.2 mmol, 12.3 ml) were added. The grey-black suspension was stirred at an oil-bath temperature of 180° C. for 16 h. After cooling to room temperature, the apparatus was rinsed with ethyl acetate (50 ml) and the reddish-brown reaction mixture was filtered through celite (15 g) which was covered with silica gel (20 g). The filter cake was rinsed with ethyl acetate (3×100 ml) and the combined filtrates were washed with 1N hydrochloric acid (3×450 ml). The aqueous phase was re-extracted with ethyl acetate (2×100 ml). The combined organic phases were washed with saturated sodium chloride solution (1×150 ml), dried over MgSO₄, filtered and concentrated and subjected to fractional distillation in vacuo and part fractions were purified by means of column chromatography. Isopropyl 4′-trifluoromethyl-2-biphenylcarboxylate was isolated in a yield of 79%. B.p.: 98-99° C./3×10⁻³ mbar. ¹H-NMR (600 MHz, CDCl₃) δ=7.92 (d, ³J=7.9 Hz, 1H), 7.66 (d, ³J=8.2 Hz, 2H), 7.52 (t, ³J=7.4 Hz, 1H), 7.40-7.46 (m, 3H), 7.31 (d, ³J=7.7 Hz, 1H), 5.01 (h, ³J=6.2 Hz, 1H), 1.04 (d, ³J=6.2 Hz, 6H) ppm. ¹³C-NMR (151 MHz, CDCl₃)=167.4, 145.4, 140.9, 131.3, 131.1, 130.3, 129.9, 129.3, 129.1, 128.8, 127.7, 125.1, 124.7 (q), 123.3, 68.5, 21.1 ppm. MS (Ion trap, EI): m/z

### 8. Preparation of 4% Trifluoromethylbiphenyl-2-carboxylic Acid

At room temperature, 50.7 g of isopropyl 4-trifluoromethylbiphenyl-2-carboxylate (0.165 mol) 200 g of N-methylpyrrolidone were introduced as initial charge in a 0.5 l sulphonation beaker with precision-ground glass stirrer and reflux condenser and admixed with 196 g of a 20% strength potassium hydroxide solution. The reaction mixture was stirred overnight at 90 to 95° C. It was then cooled to room temperature and acidified to pH 1-2 with 300 ml of a 20% strength hydrochloric acid. The reaction suspension was cooled to 10° C. and after-stirred for 3 h. The precipitate was filtered off and the filter residue was washed with copious amounts of water. The wet product was then dried overnight in a vacuum drying cabinet at 40° C. and 20 mbar. This gave 31.7 g of 4-trifluoromethylbiphenyl-2-carboxylic acid (0.119 mol, 72%) as a colourless solid.

What is claimed is:

1. Process for the preparation of the compounds of the formula (I) 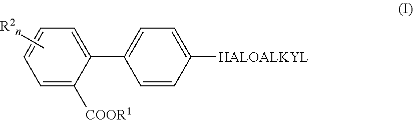
where R¹ is C₁-C₆-alkyl, C₆-C₂₄-aryl or C₇-C₁₅-arylalkyl, and R² may be independent of the others or identical and is C₁-C₆-alkyl, C₂-C₆-alkenyl, C₃-C₁₆-heteroaryl, C₁-C₆-alkoxy, C₁-C₆-aryloxy, C₁-C₆-acyl, C₇-C₁₅-arylalkyl, C₁-C₈-mono- and dialkylamino, nitro, cyano, C₁-C₆-alkylthio, C₆-C₂₄-aryl, a 3- to 7-membered saturated or partially unsaturated heterocycle or hydrogen and when R² is a C₃-heteroaryl, C₃-heteroaryl contains three carbon atoms and at least two nitrogen atoms or one nitrogen atom and one oxygen atom or one nitrogen atom and one sulphur atom,
and n=1, 2, 3 or 4,
and HALOALKYL is a straight-chain, cyclic or branched C₁-C₆-alkyl radical which is mono-, poly- or completely halogenated, i.e. substituted by F, Cl, Br and/or I,
in which compounds of the formula (II) 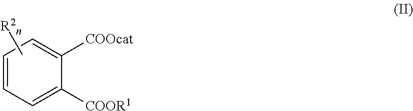
in which R¹, R² and n have the aforementioned meaning and cat is an inorganic or organic singly charged cation,
in the presence of at least one copper source,
in the presence of at least one cyclic, chelating amine,
in the presence of at least one phosphine ligand and
in the presence of at least one palladium source
are reacted with compounds of the formula (III) 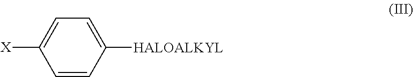
in which HALOALKYL has the aforementioned meaning and X is halogen=Cl, Br, I, pseudohalogen, arylsulphonate or alkylsulphonate, to give compounds of the formula (I).

2. Process according to claim 1, wherein R¹ is methyl, ethyl, s-propyl, n-propyl, n-, s-, tert-butyl, neopentyl, cyclohexyl, benzyl or phenyl.

3. Process according to claim 1 or 2, wherein R² is hydrogen and/or is C₁-C₆-alkyl, C₂-C₆-alkenyl, C₃-C₁₆-heteroaryl, C₆-C₂₄-aryl, C₁-C₆-acyl.

4. Process according to one or more of claims 1 to 3, wherein HALOALKYL is a C₁-C₆-perhaloalkyl, preferably a C₁-C₆-perfluoroalkyl.

5. Process according to one or more of claims 1 to 4, wherein the copper source is an inorganic copper(I) or copper(II) compound, preferably copper(I) halides, copper(II) halides or copper(II) oxide.

6. Process according to one or more of claims 1 to 5, wherein the palladium source used is an organic palladium(II) salt, preferably palladium(II) acetylacetonate.

7. Process according to one or more of claims 1 to 6, wherein the cyclic, chelating amine used is a cyclic, aromatic diamine.

8. Process according to one or more of claims 1 to 7, wherein the phosphine ligands used are compounds of the formula (IV), 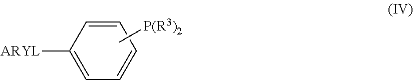
where ARYL is a C₆-C₂₄-aryl unsubstituted or substituted one or more times, independently of one another, by C₁-C₆-alkyl, C₁-C₆-alkoxy, sulphonates or C₁-C₈-mono- or dialkylamino, and R³ is a C₁-C₆-alkyl radical or a C₆-C₂₄-aryl unsubstituted or substituted one or more times.

9. Process according to one or more of claims 1 to 8, wherein X is Cl, triflate, tosylate, nonaflate or mesylate.

10. Process for the preparation of the compounds of the formula (II), 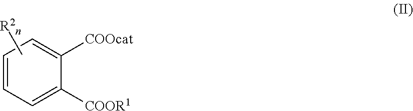
where R¹ is C₁-C₆-alkyl, C₆-C₂₄-aryl or C₂-C₁₅-arylalkyl and
R² may be independent of the others or identical and is C₁-C₆-alkyl, C₂-C₆-alkenyl, C₃-C₁₆, C₁-C₆alkoxy, C₁-C₆-aryloxy, C₁-C₆-acyl, C₇-C₁₅-arylalkyl, C₁-C₈-mono- and dialkylamino, nitro, cyano, C₁-C₆-alkylthio, C₆-C₂₄-aryl, a 3- to 7-membered saturated or partially unsaturated heterocycle or hydrogen, and when R² is a C₃-heteroaryl, the C₃-heteroaryl contains three carbon atoms and at least two nitrogen atoms or one nitrogen atom and one oxygen atom or one nitrogen atom and one sulphur atom,
and n=1, 2, 3 or 4
and cat is an inorganic or organic singly charged cation, characterized in that compounds of the formula (V) 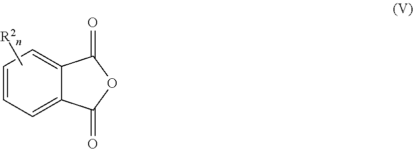
in which R² and n have the aforementioned meaning,
are reacted in a step a) with a compound of the formula (VI)
R¹(OH)ₘ (VI)
where R¹ has the aforementioned meaning and m may be 1, 2, 3 or 4,
to give compounds of the formula (VII) 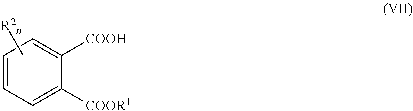
and in a step b) the compounds of the formula (VII)
are reacted with at least one base, consisting of one or more singly charged, inorganic or organic cations and corresponding anions, to give compounds of the formula (II).

11. Process according to claim 10, wherein the bases used are alkali metal hydroxides, preferably potassium hydroxide.

12. Process according to one of claim 10 or 11, wherein the compounds of the formula (II) are prepared in a one-pot reaction from the compounds of the formula (V) without isolation or purification of the compounds of the formula (VII).

13. Process according to one or more of claims 1 to 9. wherein the compounds of the formula (II) are prepared according to one or more of claims 10 to 12 and are used without isolation and further purification.

14. Process according to one or more of claims 1 to 9, wherein the compounds of the formula (VIII) 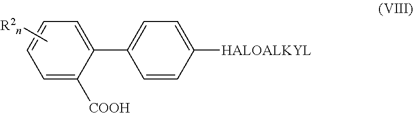
in which R², n and HALOALKYL have the meaning given for the compounds of the formula (I), are prepared by reacting the compounds of the formula (I) with an acid or a base.
